(19) United States
(12) Patent Application Publication
(10) Pub. No.: US 2003/0082227 A1
(43) Pub. Date: May 1, 2003

(54) Transdermal device comprising a reservoir and a matrix containing the same active principle

(76) Inventors: Michel Sournac, Toulouse (FR); Laurent Liorzou, L'Union (FR); Joel Bougaret, Francarville (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/148,256

(22) PCT Filed: Nov. 30, 2000
(86) PCT No.: PCT/FR00/03344

(30) Foreign Application Priority Data
Nov. 30, 1999 (FR) 99/15071

(51) Int. Cl. A61K 31/57; A61K 9/70

(52) U.S. Cl. 424/449; 514/177

(57) ABSTRACT

The invention concerns a self-adhesive transdermal device comprising at least two chambers such that the first chamber is a reservoir-type transdermal device (2) and the second chamber is a matrix-type transdermal device (6) located at the periphery of the first chamber. The invention is characterised in that said two devices contain the same active principle.

The subject of the invention is a self-adhesive device intended for the transdermal administration of an active principle, comprising a double reservoir consisting of at least one central chamber and of at least one peripheral chamber, these chambers being characterized in that they contain the same active principle. Each of these chambers exhibits a diffusion kinetic with a different and complementary profile. The invention also relates to a method for manufacturing this device and its application as a medicament.

The present invention relates to transdermal devices preferably allowing the transcutaneous administration of an active principle from at least one reservoir chamber and at least one matrix chamber, so as to combine the skin diffusion kinetics and to reduce the lag time, in order to improve the performance and the safety of the product administered. As a general rule, a reservoir chamber is located at the center of the device, whereas a matrix chamber constitutes a peripheral zone around the central part. It may also be envisaged that the matrix chamber is located in the center and that the reservoir chamber constitutes a peripheral zone around the central part. The device according to the invention is designed most particularly for active principles for which a rapid action is sought with a possible continuation in the form of an extension of the therapeutic effect. The rapid action is, for example necessary in the cases of local treatment of pain, of smoking cessation, of hormone replacement therapy in humans. The prolonged action, in each of these situations, makes it possible to prolong the effect without having recourse to another drug administration via a transdermal galenic form or the like.

Transdermal devices are pharmaceutical dosage forms which allow the percutaneous administration of certain active principles. It is recognized that this particular route for drug administration has advantages such as the avoidance of the hepatic first pass and good tolerance of the treatment by the patient. Its defects are also known, and more particularly among them, there should be mentioned the existence of a relatively long lag time. This parameter defines the interval of time between fitting the device on the skin and the first appearance of a measurable quantity of medicament in the bloodstream, attesting the passage thereof. Overcoming this drawback means administering a transdermal form according to a regular rhythm, adapted to the pathological condition treated. Finally, this defect, which is common to the transdermal devices marketed up until now, excludes any possibility of prescription once an immediate or rapid therapeutic relief is required.

This lag time depends on two factors: the capacity for an active principle to be retained by the superficial skin structures such as the stratum corneum, and also the technology chosen for producing the actual device.

Among transdermal devices, the reservoir devices are generally distinguished from the matrix devices. In reservoir devices, the active principle is contained in a gel, most often an aqueous-alcoholic gel, placed between a support film and a control membrane. In the case of devices adhering over their whole surface, a layer of a material permeable to the active principle and adhesive—or an intermediate permeation membrane—is present between the surface of the skin and the membrane for controlling the release. In the other cases, this layer is located at the periphery. In matrix devices, the active principle is contained in one or more matrix layers (generally based on self-adhesive polymers). In this case, direct contact with the skin does not at least require the presence of an intermediate permeation membrane.

In the case of a so-called reservoir technology, the lag time will be short and generally little different from that observed with a topical form of the aqueous-alcoholic gel type. In the case of a so-called matrix technology, the lag time is generally longer since the active principle in dispersed or dissolved form in an adhesive polymer has to migrate beforehand across this adhesive medium before crossing the stratum corneum.

The best example in the prior art relates to transdermal devices based on estradiol and more precisely the replacement of the original reservoir form by a matrix form. In the 1990s, a first hormone device was marketed (ESTRADERM®), in the form of a reservoir containing estradiol in the form of an alcoholic gel. A transparent protective membrane forms the reservoir proper, whereas on the side of the skin, this same reservoir is closed by a control membrane. The latter controls the release of the active principle and regulates the emptying of the reservoir. This not very esthetic device was found to be relatively unstable, most particularly during severe stress conditions where, following losses of ethanol, modifications in kinetic performance were later demonstrated.

More recently, the same laboratory has developed a replacement transdermal device (ESTRADERM MX®), by opting for a matrix form via the intimate mixing of estradiol and of adhesive compounds. This more elegant form, adhering to the whole of its surface, free of control membrane, is characterized by a much more regular rate of release. Studies (Berner B. et al., Clin. Pharmacokinet 1994, 26:121-134) have shown, in the first case, the existence of a kinetics of diffusion of estradiol which is closely dependent on that of ethanol, the principle constituent component of the gel contained in the reservoir. Thus, the gradual depletion of ethanol slows down the release of active principle. In the second case, that of a matrix form containing estradiol in solution or in dispersion in an adhesive polymer, the regulatory mechanism responsible for the diffusion of the active principle is more regular, but slower to come into action. Comparison between these two technologies (Muller et al., Eur. J. Clin. Pharmacol, 1996, 51:327-330) appears to indicate the existence of a bioequivalence limited to the extent of absorption criterion, and not extended to the rate of absorption criterion. To our knowledge, no transdermal galenic form makes it possible to combine the two modes of release, for an identical active principle, or exists in the form as proposed in this patent application.

The technical characteristics which are the most representative of the prior art are presented more particularly in patent applications WO 96/40355, WO 94/25069, WO 97/23227, WO 98/03137 or WO 92/05811. The development of the devices described in these texts remains complex and few productions have materialized to date. Indeed, in the majority of therapeutic situations, the aim is either to administer precise and different quantities of active principles from an identical adhesive composition, or to combine different release profiles from one and the same active formulation. Such formulations will have as principal causes of failure, either the instability of two active principles coexisting in the same adhesive and the possible competition at the level of their respective rates of transcutaneous passage, or the impossibility of combining two different kinetic profiles.

A second route consists in bringing together several transdermal devices each containing a single active principle. Such devices are for example described in WO 94/06383 or WO 90/06736; in this situation, it will be easier to regulate the quantity to be administered via an adjustment of the relevant surface. In return, the total surface is large and above 40-50 cm², problems of adhesion or even of skin tolerance are recognized.

Other more minor descriptions such as WO 97/47305 or WO 96/19203 may be described as follows. In the first case, adjacent matrix devices are involved which are preferably supported by the same support and protected by the same protector, but separated by a maximum distance of 10 mm. This device, which ensures the separation of two chambers, nevertheless does not make it possible to combine different modes of release, with the exception of those made possible by self-adhesive formulations. In the second case, a first chamber containing the mixture of two active principles is combined with a second chamber containing only one of the two preceding active principles. The implementation of such a description appears complex and involves the obligatory combination of two active principles, that is to say their necessarily physicochemical compatibility. It is again evident from this analysis, that to our knowledge, no transdermal galenic form makes it possible to combine the two modes of release, for an identical active principle, or exists in the form as provided in this patent application.

The solutions of the prior art are not judged very satisfactory because they do not yet constitute an ideal response to the various constraints encountered:

excessively rapid kinetics from a reservoir patch;

excessively long lag time for a matrix patch;

small total contact surface;

synergy between the diffusion techniques.

In the field of transdermal administration, it appears desirable, even in the context of a monotherapy, to have a novel technical solution which makes it possible to optimize the delivery of an active principle of which more regular immediate effects are expected.

That is what the present invention provides by virtue of the production of a device for transdermal administration with double kinetics consisting of at least one central reservoir chamber, surrounded by an adhesive crown acting as second diffusion chamber; each of these chambers being capable of releasing the same active principle according to release and diffusion characteristics which are specific to it. Among the active principles for which a double action is sought, there should be mentioned:

androgenic hormones and more especially testosterone, methyltestosterone, fluoxymesterone,

local anti-inflammatory agents and more especially piroxicam, indomethacin, ibuprofen, ketoprofen, flurbiprofen, and other local glucocorticoids;

local anesthetics and more especially lidocaine, fentanyl, tetracaine;

anxiolytics and/or antidepressants and more especially buspirone, fluoxetine, lorazepam, risperidone, selegiline;

antinauseants such as chlorpromazine, promethazine, metoclopramide, domperidone;

antiallergics and more especially chlorpheniramine, emedastine, ketotifen; mequitazine.

The subject of the present invention is a self-adhesive transdermal device for administration with double kinetics of an active principle comprising at least two adjacent chambers, which are in particular concentric, one being a reservoir-type chamber and the other being a matrix-type chamber, each of these chambers being designed to release the active principle according to release and diffusion characteristics specific to each chamber.

According to a first variant, the present invention relates to a device comprising a reservoir-type central chamber (2) surrounded by at least one matrix-type central diffusion chamber (6), and, according to a second variant, to a device which comprises a matrix-type central diffusion chamber (6a) surrounded by at least one reservoir-type chamber (2a).

The active principle is preferably an androgenic compound and still more preferably testosterone (17-hydroxandrost-4-en-3-one).

Indeed, in the context of a hormone replacement therapy (TRT for testosterone replacement therapy), it is most particularly advantageous to allow a rapid administration (which mimics the plasma concentration peaks of this hormone in healthy subjects) followed by a regular administration (which makes it possible to maintain a circulating level greater than 10 nmole/liter). It will be noted that none of the transdermal forms of testosterone marketed up until now fulfills such an objective because either they are provided in the form of a reservoir patch (rapid release of the active principle), or in the form of a matrix patch for scrotal application (rapid release linked to the low thickness of the skin of the scrotum). The present invention also relates to the use of the device according to the invention for the preparation of a medicament intended for a testosterone replacement therapy and in particular a daily, weekly or biweekly testosterone replacement therapy, for the treatment of at least one pathological condition chosen from the group consisting of the treatment and/or prevention of hypogonadism, andropause disorders, sexual impotence, fertility disorders in men, oligospermia, premature ejaculation.

According to a preferred variant of the invention, the transdermal device is such that the matrix-type chamber comprises:

a monolayer or multilayer self-adhesive matrix containing the active principle,

and optionally at least one permeation-enhancing agent, that is to say an absorption-promoting agent.

The matrix-type chamber essentially contains a self-adhesive matrix consisting of at least one self-adhesive compound chosen from the group consisting of butyl acrylate, ethyl acrylate, butyl methacrylate 2-ethylhexyl acrylate, isooctyl acrylate, acrylic acid, methacrylic acid, vinyl acetate, hydroxyethyl methacrylic acid, methyl methacrylate, methyl acrylate, EVA (ethylene-vinyl acetate), rubbery derivatives, block copolymers SIS (styrene-isoprene-styrene), SBS (styrene-butadiene-styrene), SEBS (styrene-ethylene-butylene-styrene) in combination with sticky resins such as cellophane resins, terpenic resins, hydrogenated synthetic resins and with plasticizers such as polyolefins, fatty acid esters and phthalic derivatives. These materials, which are capable of crosslinking to give a self-adhesive matrix, will be called “acrylic copolymer-type materials in the remainder of the description”.

Said matrix chamber comprises a monolayer or multilayer self-adhesive matrix. Preferably, it comprises:

a) 40 to 80 parts by weight of acrylic copolymers,

b) 5 to 20 parts by weight of a testosterone solvent chosen from:

N,N-diethyl-m-toluamide (DEET),

2-octyldodecanol,

crotamiton,

propylene glycol dipelargonate,

c) 5 to 25 parts by weight of one forming adjuvant compound chosen from:

polyvinylpyrrolidone (PVP),

xanthan gums,

cellulose derivatives such as for example sodium carboxymethyl cellulose,

d) 2 to 10 parts by weight of a testosterone-type hormone or of one of its salts.

Advantageously, there will be used for the peripheral chamber (active adhesive matrix, called AAM in the remainder of the description), an acrylic copolymer of low to medium molecular mass, having an acid function, characterized by the presence of acrylic acid among the basic monomers giving it an acid value of between 30 and 50; this copolymer having a vinyl acetate content of between 1 and 10% by weight relative to the total weight of basic monomers. This acrylic copolymer (for example DUROTAK® 387-2052 or 87-2052 from the company NATIONAL STARCH & Chemical) is a “ready-to-use” self-crosslinked adhesive available in the form of an organic solution having a theoretical density of close to 0.92 g/cm³ and a Brookfield viscosity (at 25° C., 12 rpm, rotor No. 3) in the region of 2800 mpa.s.

Among the adjuvant polymers of the matrix, which are suitable according to the invention, there may be mentioned cellulose derivatives and more particularly sodium carboxymethyl cellulose having a molecular mass of between 90,000 and 700,000 Da, alkyl cellulose-type derivatives such as hydroxyethyl cellulose or hydroxypropyl cellulose, high-molecular-weight polysaccharides and more particularly xanthan gums, 1-vinyl-2-pyrrolidone-type polymers having a molecular mass of between 2500 and 3,000,000 Da. According to the invention, polyvinylpyrrolidone will be preferably used whose better solubility in alcoholic medium facilitates dispersion in an adhesive matrix as described in the present invention.

Among the best solvents for the active principle, there will be chosen either bifunctional compounds which are also included in the composition of the solvent phase of the acrylic copolymer, such as ethanol or isopropanol, or aliphatic compounds such as N,N-diethyl-toluamide. Advantageously, the high solubility of testosterone in tetrahydrofuran as well as the compatibility of this very polar solvent with the principal components of the formulation have been demonstrated; in the context of the present invention, a solution of testosterone in THF will be preferably used in order to facilitate the mixing of the active principle in the adhesive acrylic copolymer.

According to another variant, the transdermal device according to the invention is such that the reservoir-type chamber comprises:

an aqueous-alcoholic gel comprising the active principle and at least one thickening agent, preferably such as HPC (hydroxypropyl cellulose), HPMC (hydroxypropyl methyl cellulose), HC (hydroxycellulose), carbomers,

a membrane for controlling release of the active principle,

a film for protecting the control membrane, and

optionally at least one permeation-enhancing agent.

It may in addition advantageously comprise,

e) 35 to 50 parts by weight of ethanol,

f) 46 to 50 parts by weight of water,

g) 0 to 20 parts by weight of N,N-diethyl-m-toluamide (DEET),

h) 1 to 3 parts by weight of low-molecular-weight hydroxypropyl cellulose,

i) 1.5 to 5.75 parts by weight of a testosterone- or testosterone-ester-type hormone.

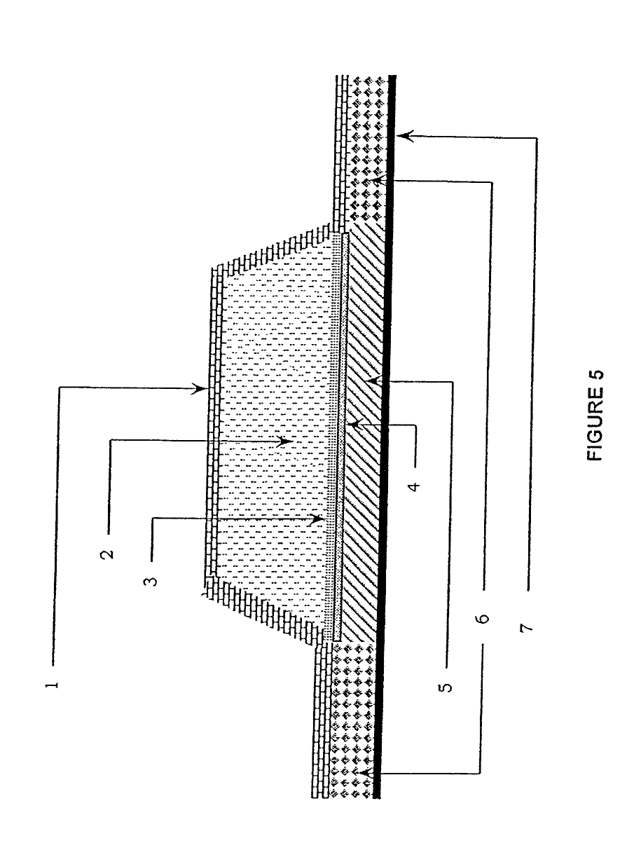

FIG. 5 represents a conventional embodiment of the transdermal device according to the invention; this figure is intended to be a nonlimiting illustration of the invention. According to this figure, the mark 1 represents a support film, the mark 2 represents a liquid active reservoir (LAR), the mark 3 represents a microporous membrane, the mark 4 represents a film for protecting the membrane, the mark 5 represents an adhesive, the mark 6 represents an active adhesive matrix (AAM) and the mark 7 represents a silicone-based protective film.

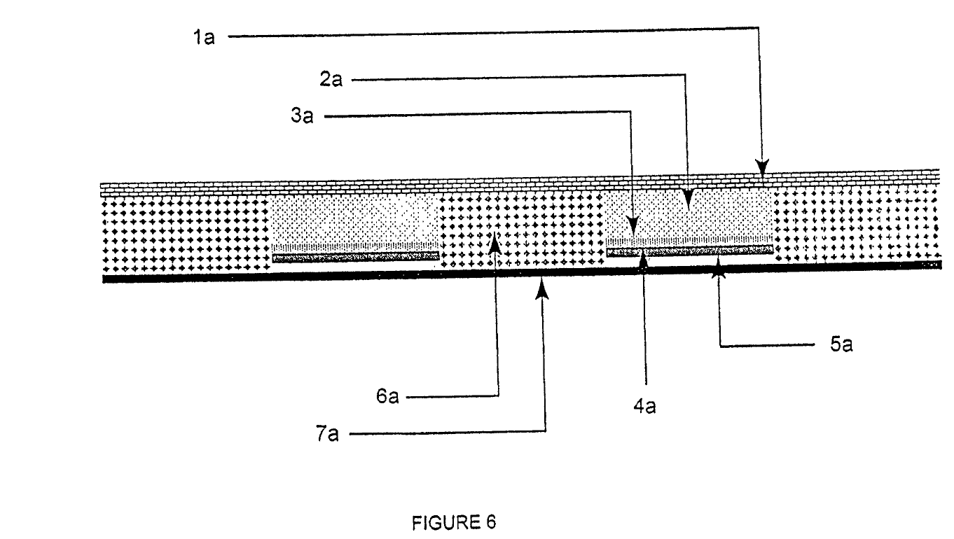

FIG. 6 represents a second embodiment of the transdermal device according to the invention. According to this embodiment, the transdermal device is composed of a matrix-type central chamber surrounded by several crowns of alternate reservoir-type and matrix-type chambers.

Provision may obviously be made for the central chamber to be a reservoir-type chamber surrounded by several crowns of alternate matrix-type and reservoir-type chambers.

FIG. 6 is a cross-sectional view of a device which comprises:

either alternate adjacent bands of reservoir-type chambers and of matrix-type chambers,

or crowns of alternate reservoir-type and of matrix-type chambers surrounding a central matrix-type chamber which has the shape of a disk.

The mark 1a represents a support film, the mark 2a represents a liquid active reservoir (LAR), the mark 3a represents a microporous membrane, the mark 4a represents a film protecting the membrane, the mark 5a represents an adhesive, the mark 6a represents an active adhesive matrix (AAM) and the mark 7a represents a silicone-based protective film.

According to the invention, a variation is also recommended which allows the presence of antioxidants, in the form of adjusted quantities, with the aim of enhancing the stability of the composition.

Preferably, the transdermal device according to the invention is such that the reservoir-type chamber comprises at least one hydrophilic-type material chosen from the group consisting of cellulose derivatives, natural gums, polyvinyl alcohols, or one hydrophobic-type material chosen from the group consisting of thermoplastic elastomers, rubbery derivatives, acrylic resins, block copolymers or alternatively combinations such as the combination of a polyacrylamide of an isoparaffin and of a polyoxyethylenated alcohol and is such that it exists in liquid, solid or semisolid form.

Advantageously, the membrane for controlling the release of the active principle from the reservoir-type chamber comprises at least one compound chosen from the group consisting of butyl acrylate, 2-ethylhexyl acrylate, isooctyl acrylate, acrylic acid, methacrylic acid, vinyl acetate, hydroxyethylmethacrylic acid, methyl methacrylate, methyl acrylate, EVA, rubbery derivatives, block copolymers SIS, SBS, SEBS, polyvinylidene fluoride, polytetrafluoroethylene, polyethylene, polypropylene, polycarbonate, polyethersulfone, cellulose esters, polyvinyl chloride, glass fibers, nylon.

Advantageously, this reservoir-type device comprises a film for protecting the membrane for controlling release of the active principle coated with an antiadherent agent, said film being a material chosen from the group consisting of silicone-based papers, silicone-based polyesters or fluorinated polyesters.

Still more preferably, the central chamber or liquid active reservoir (called LAR in the remainder of the description) will comprise a gelling agent such as low molecular weight hydroxypropyl cellulose (for example KLUCEL EF or HF® from the company AQUALON), the combination between a polyacrylamide, an isoparaffin containing 13 or 14 carbon atoms (C13-C14) and laureth 7 (for example SEPIGEL 305® from the company SEPPIC), an acrylic acid which is crosslinked via the presence of allyl sucrose and allyl ethers of pentaerythritol (for example CARBOPOL 974P® from the company BF GOODRICH). In all the preparation cases, variable proportions of water and ethanol are necessary, in respective ratios of between 1 and 3.

Among the best solvents for the active principle, there will be chosen either bifunctional compounds which are also included in the composition of the LAR such as ethanol or isopropanol, or aliphatic compounds such as N,N-diethyl-m-toluamide (DEET) or isopropyl palmitate. Advantageously, the existence of optimum proportions between the latter component and the ethanol-DEET pair, which are capable of promoting flows from the LAR has been demonstrated.

Among the androgenic components which are suitable according to the invention, there may be mentioned in particular testosterone or 17β-hydroxyandrost-4-en-3-one, as well as its salts such as acetate, enanthate, propionate, isobutyrate, undecanoate and cypionate. Testosterone will be preferably used as steroidal component.

Preferably, the device according to the invention comprises an occlusive and inert support having a thickness of between 10 and 100 μm which protects the entire device according to the invention; it may be chosen from the films most often used in the formulation of transdermal devices. Among the products generally used, there should be mentioned the mono- or multicomponent polyolefin compounds such as polyethylene, polypropylene, which are mono- or biaxially drawn, polyester-type compounds, multilayer complexes consisting of the preceding materials combined, for example with thin layers of aluminum, thermoplastic elastomer complexes of the polyurethane type or combinations of copolymers of vinyl acetate and ethylene (EVA) in the form of films or foam. Preferably, a multilayer film based on modified polyolefins and preferably based on ethylene-vinyl acetate (EVA) copolymer will be used.

The protective film which protects the entire device but which should be collected at the time of the fitting, will be preferably chosen from products having good cutting properties, inert toward the components of the LAR and of the AAM, and whose antiadherence properties are more specifically adapted in order to allow contact to be maintained between the AAM and the protector, simultaneously on removing this same protective film limited to the zone of contact with the LAR. Preferably, a polyester film will be used having a thickness of between 50 and 100 microns with an antiadherence level of between 25 and 50 N/cm.

Indeed, during the development of the transdermal device according to the invention, several trials were necessary and more particularly those intended to select the materials intended to facilitate the assembly of the two chambers. The central part or liquid active reservoir (LAR) comprises a membrane for controlling the release of the active principle, itself protected by a protective film. The characteristics of the latter are indeed specially studied in order to integrally attach this film to the protector to be removed at the time of application of the device. This phenomenon involves several forces:

an interfacial force of adhesion between the adhesive which is the principal constituent of the active peripheral part (AAM) and the support (backing) of the complete device (termed F1),

an interfacial force of adhesion between this same adhesive and the protector of the device (termed F2),

a force for rupturing the adhesive (termed F3), preferably according to a cohesive mode.

Thus, according to one of the characteristics of the invention, it has proved necessary to choose a protective film quality such that:

F1>F2, which indicates the need for the adhesive to be maintained on the support film during its separation with the protective film;

F2>>F3, which indicates the presence of a rupture of this same adhesive with respect to the film protecting the LAR membrane, limited to the periphery thereof.

As regards the absorption promoter present in either of the chambers, or even simultaneously in the LAR and the AAM, it may be chosen from the components known to persons skilled in the art which make it possible to improve the cumulative quantity and flow criteria. Preferably, a permeation-enhancing agent will be used which is chosen from the group consisting of alcohols, glycols, polyglycols, amides of the pyrrolidone type and derivatives, nonionic surfactants such as polysorbates, alkyl ethers, poloxamers, saturated or unsaturated fatty acids with a carbon chain containing from 5 to 30 carbon atoms (C₅-C₃₀), fatty alcohols, polyglycolized glycerides alone or as a mixture, glycol esters of propylene glycol or of polyglycerol, fatty acid esters of the polyol type, alkylglyceryl ether, propylene glycol, glycerine, polyoxyethylene glyceryl, sorbitan, polyoxyethylene sorbitan, polyoxyethylene glycol, sugar esters, terpenic essential oils, diethyltoluamide, crotamiton, phospholipids, lecithin derivatives, cetearyl isonononanoate, mannitan esters, xanthan gums and cellulose derivatives.

The final device (LAR+AAM) will be packaged in sealed protection either of the sachet type, or of the blister type.

The final device according to the invention has numerous advantages which may be set out as follows:

compared with the transdermal forms already comercialized, the present invention makes it possible (in the case of a testosterone-based monotherapy) to reduce the lag time (characteristic component of the reservoir technology) and to ensure a regular skin passage by virtue of a good adhesion (characteristic component of the matrix technology),

as regards the chamber called AAM, the problems of solubility of testosterone in the adhesive were solved by virtue of the use of constituents more particularly capable of solubilizing high quantities of active principle; it was found to be highly judicious to use the solvent power of organic solvents such as DEET. Consequently, it was possible to incorporate into a matrix patch a quantity of active principle per cm² which is substantially equal to that present in the reservoir patch ANDRODERM®; the present invention is characterized by the presence of a skin-patch interface which is greatly saturated with active principle, responsible for the transcutaneous passage via a passive diffusion phenomenon. Taking into account the high solubility of testosterone in the DEET solvent, it was necessary to perfectly control the manufacturing process and in particular the drying stage in order to ensure a saturation state which is essential for the operation of the matrix patch. Such a supersaturation, conventionally encountered in the formulation of transdermal devices, leads in numerous cases to an unstable physicochemical state characterized by the onset of crystallization phenomena. In the manner of patents belonging to the environment of the formulation in question, the presence of PVP considerably improved the physicochemical stability of the patch without calling into question the pharmacotechnical characteristics thereof,

as regards the chamber called LAR, the problems of solubility of testosterone in an aqueous-alcoholic gel were solved by virtue of the use of constituents which are more particularly capable of promoting the stability of testosterone. Consequently, the gelling agent which gave the best flow and consistency results is KLUCEL HF; the best solvents for the active principle, compatible with the presence of ethanol, are DEET and isopropyl palmitate in precisely defined ratios. The combination of the constituents of the LAR allow the production of a gel which is well tolerated on the skin.

The transdermal devices according to the invention are produced according to the techniques generally used by persons skilled in the art; these techniques are those of mixing, coating, drying, laminating, splitting and cutting.

The present invention also relates to a process for preparing the self-adhesive transdermal device according to the present invention comprising the stages of:

on the one hand:

1) preparing a first mixture containing an active principle and at least one permeation-enhancing agent,

2) depositing this mixture on a membrane for controlling the release of the active principle,

on the other hand,

3) preparing a premixture containing an active principle,

4) adding to the premixture obtained in stage 3 at least one material of the acrylic copolymer type, capable of crosslinking to give a self-adhesive matrix,

5) coating the mixture obtained in stage 4 over a film for protecting the membrane,

6) laminating the coated protective film of stage 5 over a transfer film and then

7) removing the transfer film from the coating obtained in stage 6 and laminating this coating over the product obtained at the end of stage 2,

an assembly is thus obtained which also has to be cut.

Advantageously, a process is also recommended for the manufacture of said transdermal device according to the following stages:

1) preparing a premixture of active principle in a solvent mixture (for example based on THF),

2) preparing a second premixture based on adhesive and polymers; (in the latter case, this involves ensuring the homogeneous dispersion of PVP, a solid finely sprayed in a ready-to-use adhesive organic solution),

3) adding to the premixture 1 the theoretical quantities of ethanol and of DEET and then incorporating the whole into the preceding adhesive preparation,

4) directly depositing the homogeneous mixture thus obtained onto a protective film, preferably of the polyester type, so as to obtain a layer having a thickness of between 50 and 150 g/m² (expressed as dry weight),

5) drying the coating thus obtained in order to evaporate the manufacturing solvents and to allow the crosslinking of the acrylic copolymer, by progressive drying at a temperature of between 50° C. and 110° C., and preferably via different modes of drying; laminating over a silicone-based transfer film,

6) the product obtained in 5 constitutes an intermediate product; it is the active adhesive matrix (AAM),

7) producing a first assembly between the control membrane and a film protecting this membrane,

8) preparing a premixture of the active principle in an aqueous-alcoholic medium in the presence of DEET,

9) incorporating a gelling agent, for example, of the KLUCEL HF type so as to obtain a homogeneous gel,

10) using a micrometering device, depositing a precise quantity of gel at the surface of the control membrane,

11) producing a second assembly by depositing on the gel a protective film so as to obtain, by sealing, a leaktight unit,

12) laminating over the film protecting the control membrane, the intermediate product obtained in 6) after having removed the transfer film.

Variants of the manufacturing process as described above are recommended, characterized by:

the production of a distinct premixture containing PVP and ethanol (stage 2),

the elimination of THF (stage 1),

the use of a short infrared-type drying mode (stage 5).

a) Manufacture of the Liquid Active Reservoir (LAR)

α) in a mixer, prepare an aqueous-alcoholic solution of testosterone; this solution does not present any particular difficulty since the proportions of each constituent have been calculated beforehand as a function of the solubility characteristics of the active principle;

β) incorporate into the mixture aα the N,N-diethyl-m-toluamide as solvent for the active principle;

γ) incorporate into the mixture aβ the gelling agent; this stage should more particularly avoid the formation of bubbles and lead to the production of a homogeneous gel having a viscosity of between 1000 and 6000 mPas; the duration of this stage is between 1 h and 3 h according to the quantities used and the rate of stirring is between 150 and 200 rpm. As a general rule, it is preferable to allow this gel to “ripen” for 10 to 12 h before passing to the next stage;

δ) mechanically assemble the polyethylene films of the COTRAN 9711® type (3M MEDICA) and silicone-based polyester films of the PET SiV1F 74H type (REXOR); at the surface of the polyethylene control membrane, deposit a precise quantity of the gel obtained in stage aγ with the aid of a metering filling device of the Microvalve TS5000 type or the like; deposit at the surface of the gel a film protecting the control membrane, of the DBLF 2050® type (DOW PLASTICS) and seal the whole, preferably with the aid of ultrasound (Soudeuse Ultrasons SONICS & Matérials—power 64%, pressure 1.5 to 2 bar).

b) Manufacture of the Active Adhesive Matrix (AAM)

α) in a mixture, prepare a solution of testosterone in tetrahydrofuran; given the high solubility of testosterone in THF which is of the order of 27% m/m, this dissolution, with stirring, presents no particular difficulty; once this premixture has been prepared, ethanol is also incorporated as manufacturing solvent, and then N,N-diethyl-m-toluamide as solvent for the active principle;

β) in a mixer-homogenizer, polyvinylpyrrolidone is gradually incorporated, with stirring, into the “ready-to-use” acrylic copolymer; this stage should lead to the production of a homogeneous mixture free of any lumps and one of the characteristics of this process is the capacity to be carried out at room temperature, which presents an advantage compared with other techniques, for example so-called “hot melt” techniques, which are generally more “stressful” toward the active ingredient;

δ) coat the homogeneous mixture thus obtained, at a temperature of between 50° C. and 110° C., over a silicone-based protective film of the PET SiV1F74HMC type (REXOR), in an amount of 50 to 150 g/m², weight measured on the dry film;

φ) laminate over a silicone-based transfer film.

c) Assembly Between LAR and AAM

α) from the intermediate product obtained in bφ, remove the transfer film and directly laminate over the intermediate product obtained in aδ;

d) the assembly thus produced should generally be subjected to final cutting stages before packaging according to either of the possible systems, sachet or blister.

The best embodiment of the invention consists in having recourse to a transdermal device whose peripheral matrix chamber contains, for a total of 100 parts by weight (all of these factors are not at all limiting but are given by way of illustration):

a) 63 to 73 parts by weight of an adhesive and self-crosslinkable acrylic copolymer in the form of a solution at about 47.5% w/v of copolymer 2-ethyl-hexyl acrylate, butyl acrylate, acrylic acid and, as crosslinking agent, aluminum acetyl acetonate, said “ready-to-use” adhesive copolymer having a glass transition temperature of the order of −50° C.;

b) 5 to 25 parts by weight of soluble polyvinyl-pyrrolidone also called polyvidone, a product obtained by polymerization of n-vinylpyrrolidone, having a density of between 400 and 550 g/l and a molecular mass preferably of between 44,000 and 54,000;

c) 9 to 15 parts by weight of N,N-diethyl-m-toluamide (DEET) or N,N-diethyl-3-methylbenzamide, as formulation agent, solvent for the active principle in the adhesive matrix;

d) 4 to 6 parts by weight of testosterone in free form.

The best embodiment of the invention consists in having recourse to a transdermal device whose central reservoir chamber contains, for a total of 100 parts by weight (all these components are not at all limiting but are given by way of illustration):

e) 35 to 50 parts by weight of ethanol;

f) 45 to 50 parts by weight of water;

g) 0 to 20 parts by weight of N,N-diethyl-m-toluamide (DEET) or N,N-diethyl-3-methylbenzamide, as formulation agent, solvent for the active principle in the reservoir;

h) 1 to 3 parts by weight of low molecular weight hydroxypropyl cellulose

i) 1.5 to 5.75 parts by weight of testosterone in free form.

### EXAMPLE 1

In a first instance, 72 g of testosterone and 271 g of tetrahydrofuran are introduced into a mixer, and stirred at room temperature for at least 15 minutes. An aliquot part of the solution obtained is isolated, that is a sample of 70 g to which 45 g of ethanol and 38 g of DEET are added. This premixture is stored until its next use.

In a mixer-homogenizer, also called transfer container, 413 g of acrylic copolymer marketed by the company NATIONAL STARCH & Company under the name DURO-TAK 387-2052® are introduced. Next, under permanent stirring and under vacuum, polyvinylpyrrolidone, marketed under the name KOLLIDON K30 by the company BASF, are gradually added. Polyvinylpyrrolidone is not easily soluble in the solvent medium for the acrylic copolymer, and at this stage of the manufacture, it is observed that the addition of the initial premixture significantly improves the capacity of polyvinyl-pyrrolidone to be homogeneously dispersed in the adhesive.

The stirring is maintained until a homogeneous mixture is obtained, and then if necessary, the mixture is left to degas for about 30 minutes.

This final mixture is transferred to the feed hopper of a coating head which makes it possible to deposit on a silicone-based polyester support a quantity of the order of 120±10 g/m². The drying is performed by applying a temperature gradient of between 50° C. and 100° C. in order to evaporate the manufacturing solvents and allow the crosslinking of the acrylic adhesive. As a general rule, it is desirable to perfectly control this stage which greatly contributes to the quality of the finished product; according to said invention, this temperature gradient is associated with the possibility of varying the method of drying, by preferably applying, in a first stage, a so-called “impacting jet hot air” mode and then in a second stage a so-called “short infrared” mode. At the end of the drying, the so-called Mat adhesive matrix, in the dry state, is laminated over a silicone-based transfer film. The rolls thus obtained are then split into intermediate products of smaller width, and stored before being assembled with the reservoir chamber.

In a second instance, 11.25 g of testosterone and 224.75 g of ethanol are introduced into a mixer and stirred at room temperature until complete dissolution is obtained. 49.90 g of N-diethyl-m-toluamide (DEET), 203.58 g of water and 10.0 g of KLUCEL HF are added to the mixer. The production of a homogeneous mixture involves the use of a mixer for a minimum of 1 h 30 min, equipped with an “anchor” type stirring shaft at a variable speed of between 100 and 1200 rpm. A final homogenization is obtained by the use of an Ultraturrax for a few minutes.

In a third stage, the polyethylene film (COTRAN 9711 type) and the silicone-based polyester film (of the PET SiV1F 74H type) are cut in the form of disks with a surface area equal to the predetermined area of contact of the central part of said device. These disks are deposited on the anvil of an ultrasound soldering device in the case of a batch manufacturing process.

In a fourth stage, a nominal quantity of the gel (intermediate product obtained from stage 2) is deposited at the center of the polyethylene film. This deposit of the order of the gram, is carried out with the aid of a precision metering-filling device. The protective film (of the DBLF 2050 type), whose attachment may be optionally improved via a chemical or physical pretreatment, is laminated over each deposit thus produced. An intermediate assembly is obtained by the application of ultrasound under conditions capable of ensuring the leaktightness of the reservoir; the Res reservoir is thus obtained.

In a fifth stage, the aim is to laminate the reservoir previously obtained to the intermediate product obtained from the first stage of manufacture, and then to carry out the final cutting of the device according to the invention.

### EXAMPLE 2

The procedure is carried out in a manner similar to example 1, by replacing the acrylic copolymer DURO-TAK 387-2052 by a self-crosslinkable acrylic copolymer, in the form of a solution containing about 28% w/v of butyl acrylate, 2-ethylhexyl acrylate, acrylic acid, 2-hydroxyethyl acrylate, methyl methacrylate and, as crosslinking agent, aluminum acetyl acetonate and t-amyl peroxypivolate, said “ready-to-use” adhesive copolymer having a glass transition temperature of −26° C. and being marketed by the company NATIONAL STARCH & Chemical under the name DURO-TAK 87-2074®.

### EXAMPLE 3

The procedure is carried out in a manner similar to example 1, by reducing the quantity of the solvent for the active principle, that is a quantity of N,N-diethyl-m-toluamide in the mixture, before coating, of the order of 6%.

### EXAMPLE 4

The procedure is carried out in a manner similar to example 1, by replacing the polyvinylpyrrolidone by another polymeric agent, preferably sodium carboxymethyl cellulose, marketed under the name CARMELLOSE® sodium by the company AQUALON.

### EXAMPLE 5

The procedure is carried out in a manner similar to example 1, by replacing the polyvinylpyrrolidone by another polymeric agent, preferably a xanthan gum, marketed under the name KELTROL CR® by the company KELCO.

### EXAMPLE 6

The procedure is carried out in a manner similar to example 1, by replacing the low-molecular-weight hydroxypropyl cellulose by another gelling agent, preferably of the hydrophilic acrylic polymer type marketed under the name CARBOPOL 974P® by the company BF GOODRICH, or of the polyacrylamide type marketed under the name SEPIGEL 305® by the company SEPPIC.

### EXAMPLE 7

The procedure is carried out in a manner similar to example 1, by replacing the acrylic copolymer DURO-TAK 387-2052 by a non-self-crosslinkable acrylic copolymer, in the form of a solution containing about 28% w/v of butyl acrylate, 2-ethylhexyl acrylate, acrylic acid, 2-hydroxyethyl acrylate, methyl methacrylate, said “ready-to-use” adhesive copolymer having a glass transition temperature of −26° C. and being marketed by the company NATIONAL STARCH & Chemical under the name DURO-TAK 87-2051®.

### EXAMPLE 8

The procedure is carried out in a manner similar to example 1, by replacing the acrylic copolymer DURO-TAK of the organic solution type by an acrylic copolymer in aqueous emulsion based on ethyl acrylate, butyl acrylate and butyl methacrylate monomers, having a viscosity of between 50 and 250 mPas and having a solid content of between 54.0 and 58.0%.

### Comparative example C1

It involves the device marketed under the trademark ANDRODERM 5 mg/day® by the company THERATECH or the version delivering only 2.5 mg/day. This reservoir device consists of an alcoholic gel of testosterone based on hydroxypropylcellulose, containing absorption-promoting agents such as methyl laurate and glycerol monooleate. The diffusive surface consisting of a control membrane is centered around a nonactive adhesive peripheral part which makes it possible to maintain the patch over a nonscrotal application zone.

### Comparative example C2

It involves the device marketed under the trademark TESTODERM TTS® by the company ALZA which delivers 5 mg/day of testosterone. This reservoir device having a large surface (60 cm²) consists of an aqueous-alcoholic gel of testosterone based on hydroxypropyl cellulose lacking absorption-promoting agents. The diffusive surface consisting of a control membrane is centered around a nonactive adhesive peripheral part which allows the patch to be maintained over a nonscrotal application zone.

Additional experiments were carried out in order to demonstrate the quantities of active agents released by a device according to the invention.

Tests 1a and 1b

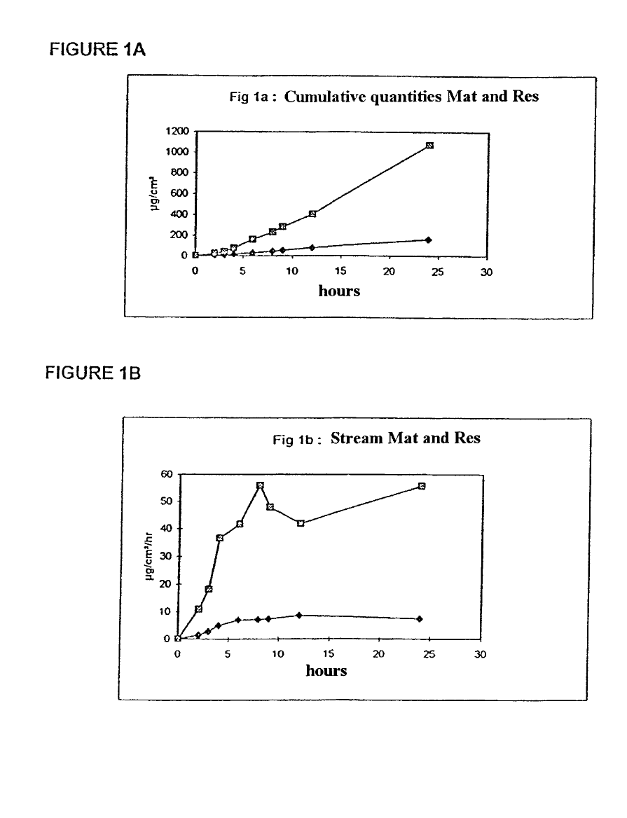

Tables 1a and 1b respectively represent the quantities of testosterone released: the permeation ex vivo on animal skins either as cumulative quantities (μg/cm²-FIG. 1a), or as stream (μg/cm²/h-FIG. 1b) as a function of time (hours), in the case of noncombined transdermal devices, that is to say either of the matrix type, or of the reservoir type.

To represent the matrix-type device, a Mat-type matrix was used and to represent the reservoir-type device, a Res-type gel was used.

The following results were obtained.

TABLE 1a

| CQ | Permeation ex viva nude mouse skins μg/cm² | | | | | | | | |
|---|---|---|---|---|---|---|---|---|---|
| h | 0 | 2 | 3 | 4 | 6 | 8 | 9 | 12 | 24 |
| Mat | 0 | 6 | 8.19 | 15 | 29.41 | 44 | 51.65 | 77.85 | 163.8 |
| Res | 0 | 21.9 | 40 | 76.51 | 160 | 232.1 | 280 | 405.8 | 1075 |

TABLE 1B

| | |
|---|---|
| Stream | Permeation ex vivo nude mouse skins μg/cm²/h |

| | | | | | | | | | |
|---|---|---|---|---|---|---|---|---|---|
| h | 0 | 2 | 3 | 4 | 6 | 8 | 9 | 12 | 24 |
| Mat | 0 | 1.4 | 2.73 | 4.9 | 7.07 | 7.2 | 7.41 | 8.73 | 7.49 |
| Res | 0 | 10.9 | 18.1 | 36.51 | 41.74 | 56.05 | 47.9 | 41.93 | 55.76 |

The results of tables 1a and 1b are represented by FIGS. 1a and 1b, respectively.

The curves joining diamonds (♦) correspond to the results obtained with the Mat matrix and the curves joining squares (▪) correspond to the results obtained with the Res device.

Tests 2a and 2b

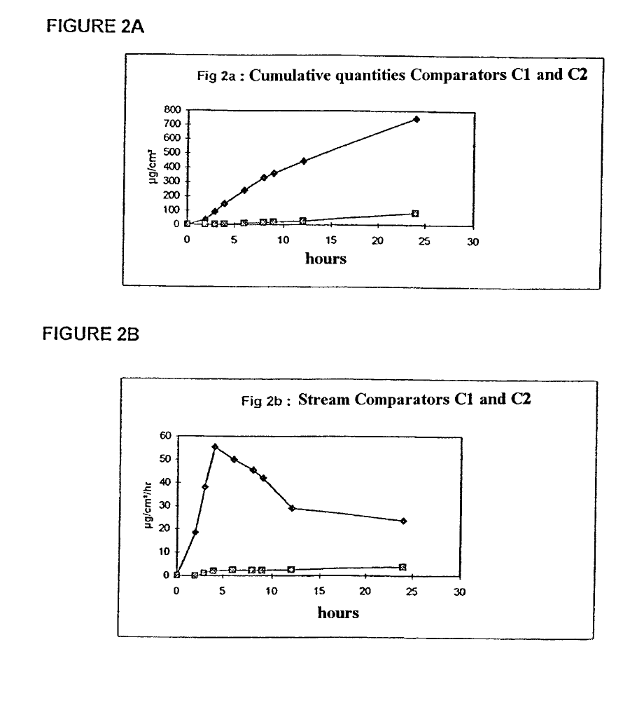

Tables 2a and 2b respectively represent the quantities released: the permeation of testosterone ex vivo on animal skins either as cumulative quantities (μg/cm²-FIG. 2a), or as stream (μg/cm²/h-FIG. 2b) as a function of time (h), in the case of reservoir-type transdermal devices, respectively as Comparator either C1 or C2.

TABLE 2a

| CQ | Permeation ex vivo nude mouse skins μg/cm² | | | | | | | | |
|---|---|---|---|---|---|---|---|---|---|
| h | 0 | 2 | 3 | 4 | 6 | 8 | 9 | 12 | 24 |
| C₁ | 0 | 37.2 | 90 | 148.1 | 240 | 330 | 360 | 445.9 | 748.6 |
| C₂ | 0 | 0 | 3.14 | 5.2 | 10.41 | 15 | 17.56 | 26.72 | 83.82 |

TABLE 2B

| | |
|---|---|
| Stream | Permeation ex vivo nude mouse skins μg/cm²/h |

| | | | | | | | | | |
|---|---|---|---|---|---|---|---|---|---|
| h | 0 | 2 | 3 | 4 | 6 | 8 | 9 | 12 | 24 |
| C₁ | 0 | 18.6 | 38 | 55.45 | 50 | 45.48 | 42 | 28.96 | 23.64 |
| C₂ | 0 | 0 | 1.04 | 2.06 | 2.42 | 2.37 | 2.4 | 2.62 | 3.84 |

The results of tables 2a and 2b are represented by FIGS. 2a and 2b, respectively.

The curves joining diamonds (♦) correspond to the results obtained with the comparative device C1 (Androderm) and the curves joining the squares (▪) correspond to the results obtained with the comparative device C2 (Testoderm).

Tests 3a and 3b

To carry out tests 3a and 3b, a Mat-type matrix and a Res-type reservoir were used. The results of these tests are presented in the table below. The quantities of testosterone released: the permeation ex vivo on animal skin in cumulative quantity (μg/cm²) as a function of time (in hours) is presented.

| CQ | Permeation ex vivo nude mouse skins μg/cm² | | | | | | | | |
|---|---|---|---|---|---|---|---|---|---|
| h | 0 | 2 | 3 | 4 | 6 | 8 | 9 | 12 | 24 |
| Mat | 0 | 6 | 8.19 | 15 | 29.41 | 44 | 51.65 | 77.85 | 163.8 |
| Res | 0 | 21.9 | 40 | 76.51 | 160 | 232.1 | 280 | 405.8 | 1075 |

Several surface combinations were made: by combining a 30 cm² matrix and a 5 cm² reservoir, a 30 cm² matrix and a 10 cm2 reservoir and then a 30 cm2 matrix and a 15 cm² reservoir. The following table presents the quantity of testosterone released as a function of time.

Summary table

| CQ | Permeation ex vivo nude mouse skins μg | | | | | | | | |
|---|---|---|---|---|---|---|---|---|---|
| h | 0 | 2 | 3 | 4 | 6 | 8 | 9 | 12 | 24 |
| Mat 30 | 0 | 180 | 245.7 | 450 | 882.3 | 1320 | 1549.5 | 2335.5 | 4914 |
| Res 10 | 0 | 219 | 400 | 765.1 | 1600 | 2321 | 2800 | 4058 | 10750 |
| Mat 30 + Res 10 | 0 | 399 | 645.7 | 1215.1 | 2482.3 | 3641 | 4349.5 | 6393.5 | 15664 |
| Res 5 | 0 | 109.5 | 200 | 382.55 | 800 | 1160.5 | 1400 | 2029 | 5375 |
| Mat 30 + Res 5 | 0 | 289.5 | 445.7 | 832.55 | 1682.3 | 2480.5 | 2949.5 | 4364.5 | 10289 |
| Res 15 | 0 | 328.5 | 600 | 1147.65 | 2400 | 3481.5 | 4200 | 6087 | 16125 |
| Mat 30 + Res 15 | 0 | 508.5 | 845.7 | 1597.65 | 3282.3 | 4801.5 | 5749.5 | 8422.5 | 21039 |

Tables 3a and 3b respectively represent the quantities of testosterone released ex vivo on animal skins in cumulative quantities (μg/cm²), either after 2 hours (table 3a) or after 24 hours (table 3b) in the case of a transdermal device having a double chamber according to the invention.

TABLE 3a
Cumulative quantities at 2 h

| Mat 30 | Mat 30 + Res 5 | Mat 30 + Res 10 | Mat 30 + Res 15 |
|---|---|---|---|
| 180 | 289.5 | 399 | 508 |

TABLE 3b
Cumulative quantities at 24 h

| Mat 30 | Mat 30 + Res 5 | Mat 30 + Res 10 | Mat 30 + Res 15 |
|---|---|---|---|
| 4914 | 10289 | 15664 | 21039 |

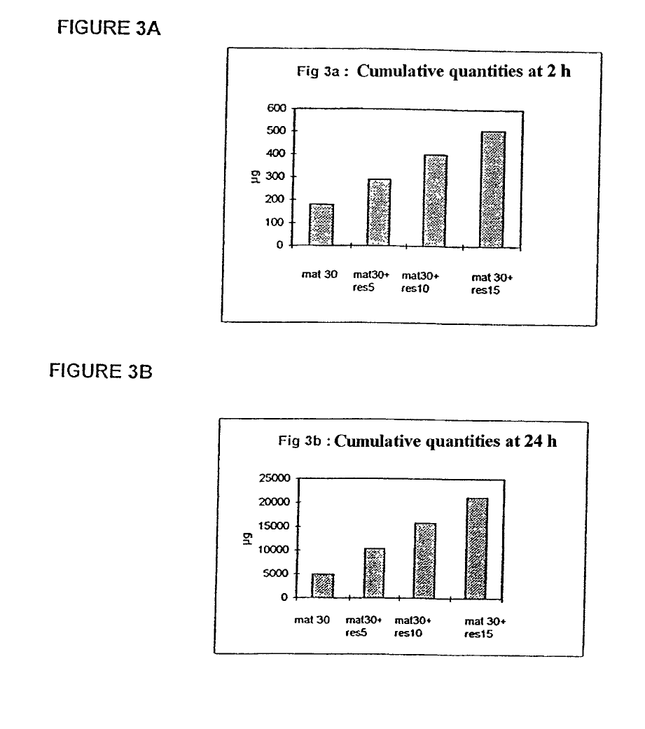

The results of tables 3a and 3b are represented by FIGS. 3a and 3b, respectively.

FIG. 3a corresponds to the cumulative quantities at 2 hours and FIG. 3b corresponds to the cumulative quantities at 24 hours.

Test 4

To carry out test 4, a Mat-type matrix and a Res-type gel were used.

The quantities of testosterone released: the permeation ex vivo on animal skin as stream (μ/cm²/h) were calculated and are presented in the table below.

| | Permeation ex vivo nude mouse skins | | | | | | | | |
|---|---|---|---|---|---|---|---|---|---|
| Stream | in μg/cm²/h | | | | | | | | |
| h | 0 | 2 | 3 | 4 | 6 | 8 | 9 | 12 | 24 |
| Mat | 0 | 1.4 | 2.73 | 4.9 | 7.07 | 7.2 | 7.41 | 8.73 | 7.49 |
| Res | 0 | 10.9 | 18.1 | 36.51 | 41.74 | 56.05 | 47.9 | 41.93 | 55.76 |

Several combinations were carried out by combining a matrix 30 and a reservoir 10, a matrix 30 and a reservoir 5, and then a matrix 30 and a reservoir 15.

The following table represents the quantities of testosterone released: permeation ex vivo on animal skin as stream (μ/unit/h).

Permeation ex vivo nude mouse skin
μ/unit/h

| | | | | | | | | | |
|---|---|---|---|---|---|---|---|---|---|
| h | 0 | 2 | 3 | 4 | 6 | 8 | 9 | 12 | 24 |
| Mat 30 | 0 | 42 | 81.9 | 147 | 212.1 | 216 | 222.3 | 261.9 | 224.7 |
| Res 10 | 0 | 109 | 181 | 365.1 | 417.4 | 560.5 | 479 | 419.3 | 557.6 |
| Mat 30 + Res 10 | 0 | 151 | 262.9 | 512.1 | 629.5 | 776.5 | 701.3 | 681.2 | 781.3 |
| Res 5 | 0 | 54.5 | 90.5 | 182.55 | 208.7 | 280.25 | 239.5 | 209.65 | 278.8 |
| Mat 30 + Res 5 | 0 | 96.5 | 172.4 | 329.55 | 420.8 | 496.25 | 461.8 | 471.55 | 503.5 |
| Res 15 | 0 | 163.5 | 271.5 | 547.65 | 626.1 | 840.75 | 718.5 | 628.95 | 836.4 |
| Mat 30 + Res 15 | 0 | 205.5 | 353.4 | 694.65 | 838.2 | 1056.8 | 940.8 | 890.85 | 1061.1 |

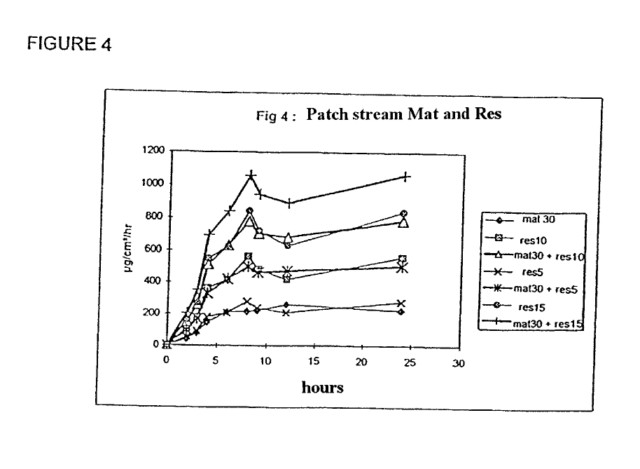

FIG. 4 represents the quantities of testosterone released ex vivo on animal skins as stream (μg/cm²/h), in the case of a transdermal device with a double chamber according to the invention.

The curve joining diamonds (♦) corresponds to the results obtained with the matrix 30, the curve joining the squares (▪) corresponds to the results obtained with the reservoir 10, the curve joining triangles (▴) corresponds to the results obtained with the combination matrix 30+reservoir 10, the curve joining the crosses (x) corresponds to the results obtained with the reservoir 5, the curve joining vertically barred crosses (*) corresponds to the results obtained with the combination matrix 30+reservoir 5, the curve joining the points (●) corresponds to the results obtained with the reservoir 15 and the curve joining vertical lines (&Rectversolid;) corresponds to the results obtained with the combination matrix 30+reservoir 15.

These tests reveal that the transdermal device according to the invention makes it possible to both reduce the lag time and to ensure a regular skin passage.

1. A self-adhesive transdermal device for administration with double kinetics of an active principle comprising at least two adjacent chambers, which are in particular concentric, one being a reservoir-type chamber and the other being a matrix-type chamber, each of these chambers being designed to release the active principle according to release and diffusion characteristics specific to each chamber.

2. The device as claimed in claim 1, such that it comprises a reservoir-type central chamber (2) surrounded by at least one matrix-type central diffusion chamber (6).

3. The device as claimed in claim 1, such that it comprises a matrix-type central diffusion chamber (6a) surrounded by at least one reservoir-type chamber (2a).

4. The device for transdermal administration as claimed in one of claims 1 to 3, characterized in that the active principle is an androgenic compound.

5. The device for transdermal administration as claimed in claim 4, characterized in that the androgenic compound is testosterone.

6. The device for transdermal administration as claimed in one of the preceding claims, characterized in that the reservoir-type chamber (2 or 2a) comprises:
an aqueous-alcoholic gel comprising the active principle and at least one thickening agent,
at least one permeation-enhancing agent,
a membrane for controlling release of the active principle (3 or 3a),
a film for protecting the control membrane (4 or 4a).

7. The device for transdermal administration as claimed in one of the preceding claims, characterized in that the matrix-type chamber (6 or 6a) comprises:
a monolayer or multilayer self-adhesive matrix containing the active principle (6 or 6a),
and at least one permeation-enhancing agent.

8. The device for transdermal administration as claimed in one of the preceding claims, characterized in that said device comprises a support (1 or 1a) on its upper part, said support being an occlusive film having a thickness of between 10 and 100 μm in a material chosen from the group consisting of mono- or multicomponent polyolefin compounds such as polyethylene, polypropylene, which are mono- or biaxially drawn, polyester-type compounds, the thermoplastic elastomer complexes of the polyurethane type or EVA.

9. The device for transdermal administration as claimed in one of the preceding claims, characterized in that the reservoir-type device (2 or 2a) comprises a film (4 or 4a) for protecting the membrane for controlling release of the active principle coated with an antiadherent agent, said film being a material chosen from the group consisting of silicone-based papers, silicone-based polyesters or fluorinated polyesters.

10. The device for transdermal administration as claimed in one of the preceding claims, such that the reservoir-type transdermal device (2 or 2a) comprises at least one hydrophilic-type material chosen from the group consisting of cellulose derivatives, natural gums, polyvinyl alcohols, or one hydrophobic-type material chosen from the group consisting of thermoplastic elastomers, rubbery derivatives, acrylic resins, block copolymers or alternatively combinations such as the combination of a polyacrylamide of an isoparaffin and of a polyoxyethylenated alcohol and is such that it exists in liquid, solid or semisolid form.

11. The device for transdermal administration as claimed in one of claims 6 to 10, characterized in that the membrane for controlling the release of the active principle (3 or 3a) from the reservoir-type chamber comprises at least one compound chosen from the group consisting of butyl acrylate, 2-ethylhexyl acrylate, isooctyl acrylate, acrylic acid, methacrylic acid, vinyl acetate, hydroxyethylmethacrylic acid, methyl methacrylate, methyl acrylate, EVA, rubbery derivatives, block copolymers SIS, SBS, SEBS, polyvinylidene fluoride, polytetrafluoroethylene, polyethylene, polypropylene, polycarbonate, polyethersulfone, cellulose esters, polyvinyl chloride, glass fibers, nylon.

12. The device for transdermal administration as claimed in one of claims 6 to 11, characterized in that the self-adhesive matrix (6 or 6a) is made from at least one self-adhesive compound chosen from the group consisting of butyl acrylate, ethyl acrylate, butyl methacrylate 2-ethylhexyl acrylate, isooctyl acrylate, acrylic acid, methacrylic acid, vinyl acetate, hydroxyethyl methacrylic acid, methyl methacrylate, methyl acrylate, EVA, rubbery derivatives, block copolymers SIS, SBS, SEBS in combination with sticky resins such as collophane resins, terpenic resins, hydrogenated synthetic resins and with plasticizers such as polyolefins, fatty acid esters and phthalic derivatives.

13. The device for transdermal administration as claimed in one of the preceding claims, characterized by the presence, in at least one of the chambers (2 or 2a) (6 or 6a), of at least one permeation-enhancing agent chosen from the group consisting of alcohols, glycols, polyglycols, amides of the pyrrolidone type and derivatives, nonionic surfactants such as polysorbates, alkyl ethers, poloxamers, saturated or unsaturated fatty acids with a carbon chain containing from 5 to 30 carbon atoms (C₅-C₃₀), fatty alcohols, polyglycolized glycerides alone or as a mixture, glycol esters of propylene glycol or of polyglycerol, fatty acid esters of the polyol type, alkylglyceryl ether, propylene glycol, glycerine, polyoxyethylene glyceryl, sorbitan, polyoxyethylene sorbitan, polyoxyethylene glycol, sugar esters, terpenic essential oils, diethyltoluamide, crotamiton, phospholipids, lecithin derivatives, cetearyl isonononanoate, mannitan esters, xanthan gums and cellulose derivatives.

14. A method for preparing the self-adhesive device for transdermal administration as claimed in one of the preceding claims, characterized in that it comprises the stages of:
on the one hand:
1) preparing a first mixture containing an active principle and at least one permeation-enhancing agent,
2) depositing this mixture on a membrane for controlling the release of the active principle (3 or 3a),
on the other hand:
3) preparing a premixture containing an active principle,
4) adding to the premixture obtained in stage 3 at least one material of the acrylic copolymer type, capable of crosslinking to give a self-adhesive matrix (6 or 6a),
5) coating the mixture obtained in stage 4 over a film for protecting the membrane (4 or 4a),
6) laminating the coated protective film of stage 5 over a transfer film and then
7) removing the transfer film from the coating obtained in stage 6 and laminating this coating over the product obtained at the end of stage 2,
an assembly is thus obtained which also has to be cut.

15. The use of the device as claimed in any one of claims 1 to 13, for the preparation of a medicament intended for a testosterone replacement therapy, preferably a daily, weekly or biweekly testosterone replacement therapy.

16. The use as claimed in claim 15, characterized in that the medicament is intended for the treatment and/or prevention of hypogonadism.

17. The use as claimed in claim 15, characterized in that the medicament is intended for the treatment and/or prevention of andropause disorders.

18. The use as claimed in claim 15, characterized in that the medicament is intended for the treatment and/or prevention of sexual impotence.

19. The use as claimed in claim 15, characterized in that the medicament is intended for the treatment and/or prevention of fertility disorders in men.

20. The use as claimed in claim 15, characterized in that the medicament is intended for the treatment and/or prevention of oligospermia.

21. The use as claimed in claim 15, characterized in that the medicament is intended for the treatment and/or prevention of premature ejaculation.
